(19) United States
(12) Patent Application Publication
(10) Pub. No.: US 2023/0042067 A1
(43) Pub. Date: Feb. 9, 2023

(54) USER-MOUNTABLE ELECTRONIC DEVICE WITH DEPLOYMENT GUIDANCE FEATURES

(71) Applicant: MEDTRONIC MINIMED, INC., Northridge, CA (US)

(72) Inventors: Jesse Hefner, Minneapolis, MN (US); Ellis Garai, Woodland Hills, CA (US); Claire F. Ferraro, Plymouth, MN (US); Justin L. Snesrud, Chaska, MN (US); Michael J. Brown, Coon Rapids, MN (US)

(21) Appl. No.: 17/397,664

(22) Filed: Aug. 9, 2021

(51) Int. Cl. G06F 1/16; A61B 5/00; A61B 5/145; G01P 15/02; G01C 19/02; G01R 33/02

(52) U.S. Cl. CPC G06F 1/163; A61B 5/0002; A61B 5/14532; A61B 5/681; G01P 15/02; G01C 19/02; G01R 33/02; G06F 3/011

(57) ABSTRACT

The disclosed system includes a user-mountable electronic device, an output interface, and at least one processor. The electronic device includes a housing and at least one sensor device located within the housing and configured to generate sensor output that indicates orientation or motion of the user-mountable electronic device. The at least one processor is operated to: receive the sensor output; identify, based on the received sensor output, a body part on which the user intends to deploy the user-mountable electronic device; determine a preferred orientation of the user-mountable electronic device relative to the identified body part; and cause the output interface to provide deployment guidance that indicates the preferred orientation of the user-mountable electronic device.

### TECHNICAL FIELD

The present technology is generally related to an electronic device that is affixed to, mounted to, inserted into, or otherwise deployed onto the body of a user. More specifically, the present technology relates to the use of at least one sensor (such as an accelerometer) onboard a user-mountable electronic device for purposes of providing guidance to the user when deploying the electronic device onto the body.

### BACKGROUND

A variety of personal or portable electronic devices are intended for application to the body or skin of a user. For example, electronic medical devices (such as physiological characteristic or analyte sensors, monitor devices, fluid infusion pumps, and activity monitors) can be designed as user-mounted or user-attached devices. In this regard, analyte sensors have been designed for use in obtaining an indication of blood glucose (BG) and subcutaneous sensor glucose (SG) levels and monitoring glucose levels in a diabetic patient, with the distal segment portion of the sensor positioned subcutaneously in direct contact with patient extracellular fluid.

A continuous glucose monitor (CGM) device with a glucose sensor of the type described above may be packaged and sold as an assembled product that includes certain features or components that allow the patient to position, deploy, and subcutaneously implant the sensor. For example, glucose sensors are often subcutaneously/transcutaneously implanted using a needle that punctures the skin of the patient as the sensor is deployed and forced onto the body. The glucose sensor device is compatible with an deployment or insertion device that cooperates with an insertion needle to insert the sensor.

Some user-mountable electronic devices, including CGM devices and “patch pump” style fluid infusion devices, may be sensitive to the particular deployment location on the body of the user and/or the particular orientation of the device relative to the body part on which it is deployed. For example, it may be desirable to deploy a CGM device on a certain body part (e.g., the thigh or the abdomen) and in a certain physical orientation or layout. Preferred mounting locations and/or device orientations may be more convenient, result in better outcomes, provide better user comfort, or the like.

Accordingly, it is desirable to have a low-cost and reliable mechanism and methodology that guides or instructs a user during deployment of a user-mountable electronic device. Furthermore, other desirable features and characteristics will become apparent from the subsequent detailed description and the appended claims, taken in conjunction with the accompanying drawings and the foregoing technical field and background.

### BRIEF SUMMARY

The subject matter of this disclosure generally relates to user-mountable devices, such as a device with a continuous glucose monitoring (CGM) sensor that is inserted into the subcutaneous tissue. The device (or an assembly that includes the device) has at least one onboard orientation, motion, movement, or acceleration sensor, such as an accelerometer. The sensor output is processed to identify a body part on which the device will be deployed, and to provide a preferred or recommended orientation of the device (relative to the identified body part). Appropriate guidance or user instructions are output to a user device to assist the user during deployment of the device onto the body.

In one aspect, the present disclosure provides a system having a user-mountable electronic device that includes a housing and at least one sensor device located within the housing and configured to generate sensor output that indicates orientation or motion of the user-mountable electronic device. The system also has an output interface and at least one processor. The at least one processor is operated to: receive the sensor output generated by the at least one sensor device; identify, based on the received sensor output, a body part on which the user intends to deploy the user-mountable electronic device; determine a preferred orientation of the user-mountable electronic device relative to the identified body part; and cause the output interface to provide deployment guidance that indicates the preferred orientation of the user-mountable electronic device.

In another aspect, the disclosure provides a method that involves: receiving, from at least one sensor device of a user-mountable electronic device, sensor output that indicates orientation or motion of the user-mountable electronic device; identifying, based on the received sensor output, a body part on which the user intends to deploy the user-mountable electronic device; determining a preferred orientation of the user-mountable electronic device relative to the identified body part; and causing an output interface to provide deployment guidance that indicates the preferred orientation of the user-mountable electronic device.

In another aspect, the disclosure provides a computer-based system that includes: computer-readable storage media to store program code instructions; and at least one processor. The program code instructions are configurable to cause the at least one processor to perform a method that involves the steps of: receiving, from at least one sensor device of a user-mountable electronic device, sensor output that indicates orientation or motion of the user-mountable electronic device; identifying, based on the received sensor output, a body part on which the user intends to deploy the user-mountable electronic device; determining a preferred orientation of the user-mountable electronic device relative to the identified body part; and causing an output interface to provide deployment guidance that indicates the preferred orientation of the user-mountable electronic device.

In another aspect, the disclosure provides a method that involves: receiving, from at least one sensor device of a first user-mountable electronic device, sensor output that indicates orientation or motion of the first user-mountable electronic device, and corresponding performance data for the first user-mountable device; identifying, based on the received sensor output and corresponding performance data, a preferred body part or orientation for deployment of a second user-mountable electronic device; and causing an output interface to provide deployment guidance that indicates the preferred body part or orientation for deployment of the second user-mountable electronic device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a simplified block diagram representation of an exemplary embodiment of a system that includes a user-mountable device or assembly having at least one onboard sensor device;

FIG. 2 is a block diagram representation of a user-mountable electronic device and a compatible deployment device, prior to deployment of the electronic device onto the body of the user;

FIG. 3 is a block diagram representation of the user-mountable electronic device and the deployment device of FIG. 2, during deployment of the user-mountable electronic device onto the body of the user;

FIG. 4 is a block diagram representation of the user-mountable electronic device of FIG. 2, after deployment onto the body of the user;

FIG. 5 is a schematic side view of an embodiment of a user-mountable analyte sensor device, after deployment and attachment to the body of the user;

FIG. 6 is a side perspective view of an embodiment of a user-mountable device implemented as an analyte sensor combined with a fluid infusion set;

FIG. 7 is a top perspective view of an embodiment of a user-mountable device implemented as a patch pump device that is suitable for use as an insulin delivery system;

FIG. 8 is a block diagram representation of an embodiment of a computer-based or processor-based device suitable for deployment in the system shown in FIG. 1; and

FIG. 9 is a flow chart that illustrates an embodiment of a device deployment guidance process.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Program code instructions may be configurable to be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

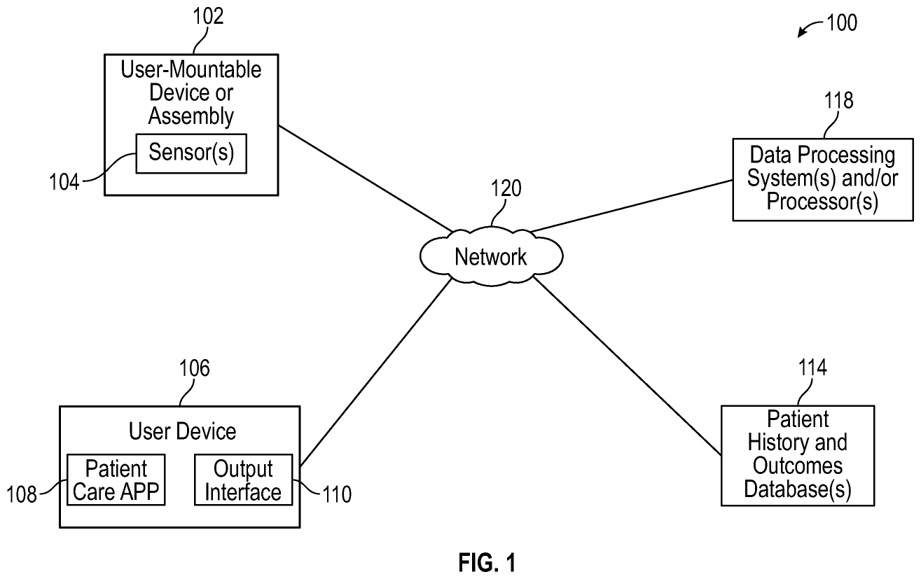

FIG. 1 depicts an exemplary embodiment of a system 100 that is compatible with a user-mountable electronic device or assembly 102. Certain embodiments of the system 100 include, without limitation: the user-mountable electronic device or assembly 102; at least one user device 106 that includes or cooperates with a suitably written and configured patient care application 108; an output interface 110 (which may be implemented at the user device 106, as shown, and/or at another device accessible to the user); at least one patient history and outcomes database 114, and at least one data processing system and/or processor 118, which may be in communication with any or all of the other components of the system 100. Other configurations and topologies for the system 100 are also contemplated here, such as a system that includes additional intermediary, interface, or data repeating devices in the data path between a sending device and a receiving device.

At least some of the components of the system 100 are communicatively coupled with one another to support data communication, signaling, and/or transmission of control commands as needed, via at least one communications network 120. The at least one communications network 120 may support wireless data communication and/or data communication using tangible data communication links. FIG. 1 depicts network communication links in a simplified manner. In practice, the system 100 may cooperate with and leverage any number of wireless and any number of wired data communication networks maintained or operated by various entities and providers. Accordingly, communication between the various components of the system 100 may involve multiple network links and different data communication protocols. In this regard, the network can include or cooperate with any of the following, without limitation: a local area network; a wide area network; the Internet; a personal area network; a near-field data communication link; a cellular communication network; a satellite communication network; a video services or television broadcasting network; a network onboard a vehicle; or the like. The components of the system 100 may be suitably configured to support a variety of wireless and wired data communication protocols, technologies, and techniques as needed for compatibility with the at least one communication network 120.

As explained in more detail below, the user-mountable electronic device or assembly 102 includes at least one sensor device 104 that is configured, arranged, and operated to generate sensor output that indicates orientation, motion, velocity, acceleration, and/or other physical status of the user-mountable electronic device or assembly 102. To this end, the at least one sensor device 104 may include or be realized as one or more of the following, without limitation: an accelerometer (single-axis, multiple-axis, etc.); an inertial measurement unit (IMU), which may be an electronic device that measures an object's specific force, angular rate, orientation, and/or other characteristics, typically using a combination of accelerometers, gyroscopes, magnetometers, etc.; a gyroscope; a magnetometer; a gravimeter; a global positioning system unit; or the like.

The system 100 can support any number of user devices 106 that are linked to, used by, owned by, or otherwise associated with the particular user or patient. In this regard, a user device 106 may be, without limitation: a smartphone device; a laptop, desktop, or tablet computer device; a medical device; a wearable device; a global positioning system (GPS) receiver device; a system, component, or feature onboard a vehicle; a smartwatch device; a television system; a household appliance; a video game device; a media player device; or the like. For the example described here, the user-mountable electronic device or assembly 102 and the at least one user device 106 are owned by, operated by, or otherwise linked to the same user/patient. Any given user device 106 can host, run, or otherwise execute the patient care application 108. In certain embodiments, for example, the user device 106 is implemented as a smartphone with the patient care application 108 installed thereon. In accordance with another example, the patient care application 108 is implemented in the form of a website or webpage, e.g., a website of a healthcare provider, or a website of the manufacturer, supplier, or retailer of the user-mountable electronic device or assembly 102. In accordance with another example, the user-mountable electronic device or assembly 102 executes the patient care application 108 as a native function.

In accordance with the illustrated embodiment, the output interface 110 resides at the user device 106. Alternatively or additionally, an output interface 110 may reside elsewhere in the system 100. For example, an output interface 110 may be deployed at the user-mountable device or assembly 102 and/or at one or more additional or supplemental electronic devices that are supported by the system 100. The output interface 110 is suitably configured, controlled, and operated to generate and provide suitably formatted output, content, or information to the user. In this regard, the output interface 110 may include or cooperate with one or more of the following, without limitation: a display element; a touch screen; an audio transducer (e.g., a speaker); indicator light(s); a haptic feedback element; or the like. The output interface 110 may be utilized to generate, present, render, output, and/or annunciate alerts, alarms, messages, images, video clips, or notifications as needed. For example, the output interface 110 can be controlled and operated to provide deployment guidance that indicates a preferred body part for deployment of the user-mountable device or assembly 102, that indicates a preferred deployment orientation of the user-mountable device or assembly 102, and/or that indicates user instructions for deploying the user-mountable device or assembly 102. The deployment guidance may be conveyed in the form of an animated display, one or more images, a video clip, written instructions, audio instructions, etc. The deployment guidance may be generated and provided in real-time or substantially real-time in response to sensor output generated by the at least one sensor device 104 while the user prepares the user-mountable electronic device or assembly 102 for deployment and/or during the deployment operation. Alternatively or additionally, the deployment guidance may be generated and provided in non-real-time in response to historical sensor output collected from one or more previously deployed user-mountable electronic devices or assemblies over a designated period of time, e.g., several days, a week, a month, etc. In some examples, the historical sensor output may include sensor performance data and corresponding to historic deployment data (e.g., body part and/or orientation information).

The at least one processor 118 may be implemented or performed with a general purpose processor, a content addressable memory, a microcontroller unit, a digital signal processor, an application specific integrated circuit, a field programmable gate array, any suitable programmable logic device, discrete gate or transistor logic, discrete hardware components, or any combination designed to perform the functions described here. Moreover, the at least one processor 118 may be implemented as a combination of computing devices, e.g., a combination of a digital signal processor and a microprocessor, a plurality of microprocessors, one or more microprocessors in conjunction with a digital signal processor core, or any other such configuration. For ease of illustration, FIG. 1 depicts the at least one processor 118 as a separate block. In practice, however, the at least one processor 118 may be located at the user-mountable device or assembly 102, at the user device 106, at a cloud-based remote computing device (e.g., a server system), and/or at another component or system that can communicate via the network 120. In certain embodiments, the at least one processor is located at a computer-based device other than the user-mountable electronic device or assembly 102.

In certain implementations, the system 100 includes a medication delivery system (not separately shown in FIG. 1). In this regard, the user-mountable device or assembly 102 may include or be realized as a medication delivery system. Alternatively or additionally, the user device 106 may include or be realized as a medication delivery system. Alternatively or additionally, a distinct medication delivery system may be included in the system 100 as another component that communicates with the network 120. For example, the medication delivery system may be realized as a user-activated or user-actuated fluid delivery device, such as a manual syringe, an injection pen, a smart insulin pen, or the like. As another example, the medication delivery system may be implemented as an electronic device that is operated to regulate the delivery of medication fluid to the user. In certain embodiments, however, the medication delivery system includes or is realized as an insulin infusion device, e.g., a portable patient-worn or patient-carried insulin pump, a smart insulin pen, or the like. In such embodiments, the user-mountable device or assembly 102 may include or be realized as a glucose meter, a glucose sensor, or a continuous glucose monitor. For the sake of brevity, conventional techniques related to insulin infusion device operation, infusion set operation, and other functional aspects of the systems (and the individual operating components of the systems) may not be described in detail here. Examples of infusion pumps may be of the type described in, but not limited to, U.S. Pat. Nos. 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893; each of which are herein incorporated by reference.

The at least one patient history and outcomes database 114 may include historical data related to the user's physical condition, physiological response to medication regulated by the medication delivery system, activity patterns or related information, eating patterns and habits, work habits, and the like. Furthermore, the database 114 may be maintained and operated such that it includes preferred orientations for user-mountable electronic devices, and for different body part deployments. Preferred deployments and device orientations may result in: better device performance (e.g., sensor accuracy); increased device lifespan; improved wireless connectivity or wireless performance; improved user comfort; better patient outcomes; improved appearance or aesthetics; or the like. The preferred orientation data may be personal to the particular user, and based on historical device deployments and corresponding results, medical outcomes, and the like. Alternatively or additionally, the preferred orientation data may include data collected for a population of users (other than the particular user of the system 100). Although preferred device orientation usually applies to physically asymmetric devices, it may also apply to symmetric devices (e.g., round or circular devices) having operating characteristics or functionality that may be impacted by the device orientation.

In accordance with embodiments where the medication delivery system is an insulin infusion device, the database 114 can maintain any of the following, without limitation: historical glucose data and corresponding date/time stamp information; insulin delivery and dosage information; user-entered stress markers or indicators; device orientation data (provided by the at least one sensor device 104) and corresponding date/time stamp information; diet or food intake history for the user; location information; and/or any other information that may be generated by or used by the system 100.

A patient history and outcomes database 114 may reside at the user device 106, at a medication delivery system, at a data processing system, or at any network-accessible location (e.g., a cloud-based database or server system). In certain embodiments, a patient history and outcomes database 114 may be included with the patient care application 108. The patient history and outcomes database 114 enables the system 100 to generate recommendations, warnings, and/or device deployment guidance for the user. The database 144 may also enable the system 100 to regulate the manner in which the medication delivery system functions to administer therapy to the user, based on the detected sensor output (generated by the at least one sensor device 104).

The following description relates to methodologies for deploying a user-mountable electronic device, such as a medical device or component, that is designed to be affixed to the body of a user. In this regard, the electronic device is deployable onto a body part of the user via, for example, a suitably configured deployment device that is compatible with the electronic device. Referring to FIG. 1, the user-mountable electronic device or assembly 102 may be an assembly that includes the mountable device and its compatible deployment device (wherein a sensor device 104 may reside in the mountable device and/or in the deployment device). The electronic device is placed and affixed onto the body of the user during a deployment action using, e.g., a compatible deployment device that quickly moves the electronic device toward the body. In accordance with the exemplary embodiment contemplated here, the electronic device has an onboard accelerometer (e.g., the sensor device 104 of FIG. 1) to measure or detect acceleration, movement, orientation, or motion of the electronic device during deployment. In this regard, the accelerometer provides output while the user holds, handles, manipulates, and positions the device during the deployment routine. In addition, the accelerometer can provide output before, during, and after the deployment device is actuated.

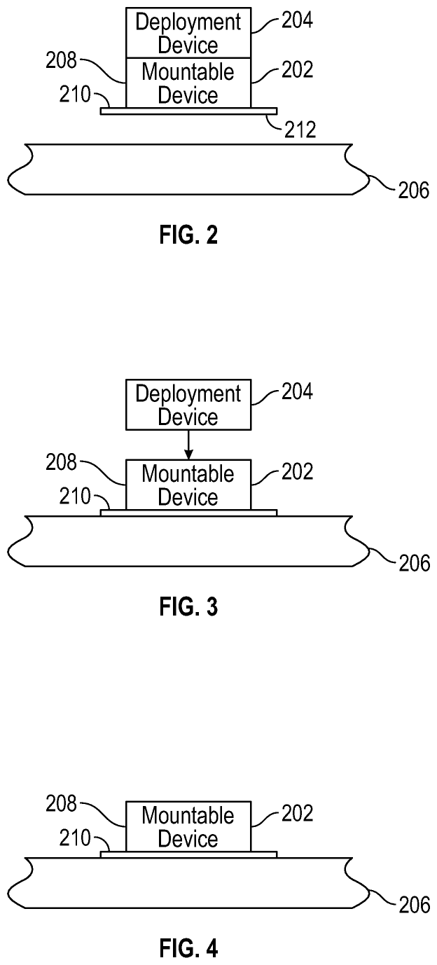

FIG. 2 is a block diagram representation of a user-mountable electronic device 202 and a compatible deployment device 204, prior to deployment of the electronic device 202 onto a body part 206 of the user. FIG. 3 is a block diagram representation of the electronic device 202 and the deployment device 204, during deployment of the electronic device 202 onto the body part 206 of the user, and FIG. 4 is a block diagram representation of the electronic device 202 after deployment onto the body part 206 of the user. The electronic device 202 includes a housing 208 that covers, encloses, or otherwise protects its internal components. The electronic device 202 may include an adhesive patch 210 (or any suitably configured adhesive element or component) on a bottom surface of the housing 208. The adhesive patch 210 may be provided with a removable liner (not shown) that is removed to reveal an exposed adhesive surface 212 of the adhesive patch 210, as depicted in FIG. 2. In certain embodiments, the electronic device 202 and the deployment device 204 are coupled together as a unit before actuation of the deployment device (see FIG. 2). In this regard, the electronic device 202 and the deployment device 204 can be manufactured, assembled, and packaged as a combined unit that is appropriate for storage, shipping, and handling. Alternatively, the electronic device 202 and the deployment device 204 can be individually offered as separate (but compatible) components that require assembly or loading before deployment of the electronic device 202 onto the body part 206.

As schematically depicted by the downward pointing arrow in FIG. 3, actuation, triggering, or activation of the deployment device 204 moves the user-mountable electronic device 202 toward the body part 206 of the user. The deployment device 204 includes at least one spring, a pneumatic feature, a pre-tensioned structure, or equivalent structure that imparts downward force to the electronic device 202 when the deployment device 204 is actuated. For example, the deployment device 204 and the electronic device 202 may be provided in a pre-loaded state that is ready to deploy, or it may be necessary for the user to manipulate the deployment device 204 and/or the electronic device 202 into a loaded state before deployment.

Actuation of the deployment device 204 releases the electronic device 202 such that it moves toward the body part 206 with a deployment acceleration that is sufficient to adhere the adhesive surface 212 (of the adhesive patch 210) to the body part 206. If the electronic device 202 includes an insertable analyte sensor, an insertable fluid delivery cannula, and/or another body-insertable element, then the deployment acceleration is also sufficient to facilitate insertion of such insertable element(s) into the body part 206. FIG. 4 shows the electronic device 202 by itself after deployment onto the body part 206. The deployment device 204 is decoupled and removed from the electronic device 202 after it has been actuated. In certain embodiments, the deployment device 204 retains one or more insertion needles that retract after insertion of at least one body-insertable element (such as an analyte sensor and/or a fluid delivery cannula). Accordingly, the deployment device 204 may be discarded after the electronic device 202 has been attached to the body part 206.

In accordance with certain embodiments, the user-mountable electronic device or assembly 102 includes or is realized as an analyte sensor that measures a physiological characteristic of the user, such as glucose. In accordance with some embodiments, the user-mountable electronic device or assembly 102 is realized as a continuous glucose monitor having at least one glucose sensor. In such embodiments, the system 100 may include or cooperate with a physically distinct insulin infusion device (e.g., a pump with a tethered insulin infusion set, a user-activated insulin pen, a patch pump, or the like) that can receive the sensor output from the user-mountable electronic device for appropriate handling, processing, and/or routing.

In accordance with some embodiments, the user-mountable electronic device or assembly 102 is realized as a single component that includes: (1) an analyte sensor that measures a physiological characteristic of the user, such as glucose; and (2) a medicament delivery element, such as a fluid delivery cannula, conduit, or needle. In accordance with some embodiments, the user-mountable electronic device or assembly 102 is realized as a single component that includes: (1) an analyte sensor that measures a physiological characteristic of the user, such as glucose; (2) a medicament delivery element, such as a fluid delivery cannula, conduit, or needle; and (3) a fluid pump mechanism or system that is electronically controlled to regulate the delivery of a medicament to the user via the fluid delivery cannula, conduit, or needle. In accordance with some embodiments, the user-mountable electronic device or assembly 102 includes or is realized as a fluid infusion device, such as an insulin infusion pump. In accordance with some embodiments, the user-mountable electronic device or assembly 102 includes or is realized as a medication fluid infusion set having a body-insertable fluid conduit.

Accordingly, the user-mountable electronic device or assembly 102 may be a component of a system used to treat diabetes (such as, a continuous glucose sensor with wireless communication capability, an insulin infusion device, or a blood glucose meter/monitor), although embodiments of the disclosed subject matter are not so limited. For the sake of brevity, conventional features and characteristics related to infusion systems, analyte sensors such as continuous glucose sensors, and fluid conduits such as soft cannulas may not be described in detail here.

The user-mountable electronic device or assembly 102 may include one or more elements or components that are intended for insertion into or application onto the body of the user. For example, the electronic device or assembly 102 may include an analyte sensor that is designed to be inserted into the skin of the user. In certain embodiments, the electronic device or assembly 102 includes or is realized as a continuous glucose monitor having a glucose sensor that is insertable into the user to obtain sensor glucose measurements. Accordingly, actuation of the deployment device initiates insertion of the analyte sensor into the user. As another example, the electronic device or assembly 102 may be implemented as a fluid infusion device having a fluid delivery cannula or needle that is designed to be inserted into the skin of the user. In certain embodiments, the electronic device or assembly 102 includes a fluid delivery cannula that is insertable into the user to deliver insulin from a fluid reservoir to the user. Accordingly, actuation of the deployment device initiates insertion of the fluid delivery cannula or needle into the user.

In certain embodiments, the user-mountable electronic device or assembly 102 is realized as (or includes) a transdermal medicament delivery device that is configured to deliver a medicament to the user through the skin without using an inserted needle, cannula, or conduit. Similarly, the user-mountable electronic device or assembly 102 may be realized as (or include) a transdermal analyte sensor device that is configured to detect or measure a physiological characteristic of the user through the skin or via contact with the skin, without using an inserted sensor element, electrode, or lead. As additional examples, the user-mountable electronic device or assembly 102 may include or be realized as any of the following, without limitation: a body temperature monitor; a heartrate monitor; a pulse oximeter; a sweat sensor; and/or an activity tracker. A user-mountable electronic device of this type may include an adhesive strip or component that secures the device to the skin of the user after deployment. In some embodiments, the user-mountable electronic device is realized as an implantable device that is injected or implanted into the body of the user via the application of force that results in a detectable deployment acceleration.

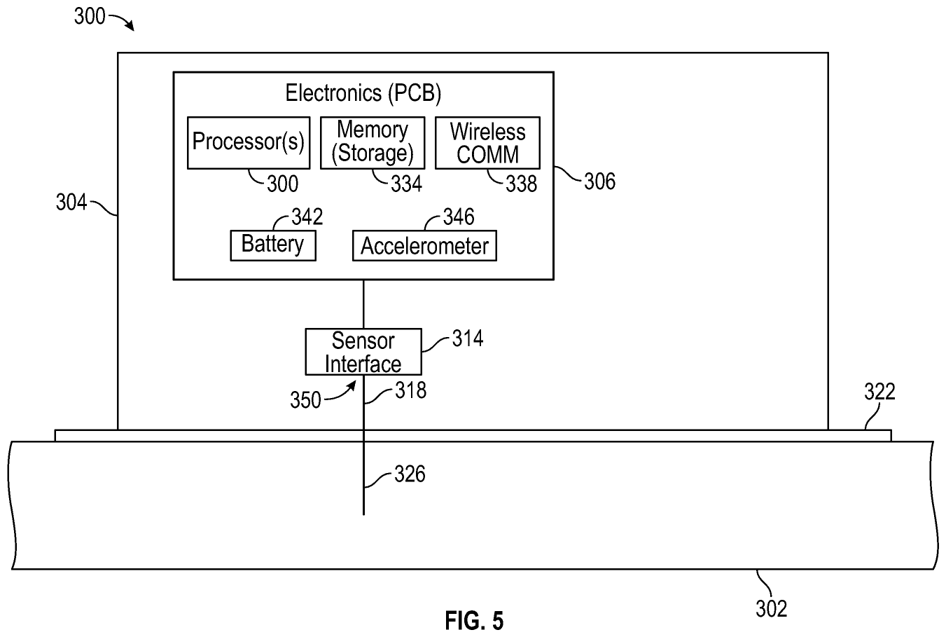

FIG. 5 is a schematic side view of an embodiment of a user-mountable analyte sensor device 300, after deployment and attachment to a body part 302 of the user. The sensor device 300 is one suitable implementation of the user-mountable electronic device or assembly 102 shown in FIG. 1. In accordance with certain embodiments, the sensor device 300 is realized as a continuous glucose monitor having a glucose sensor element (e.g., an analyte sensor 318). FIG. 5 depicts the housing 304 of the sensor device 300, along with various components, elements, and devices that are housed by, enclosed within, or attached to the housing 304. It should be appreciated that an embodiment of the sensor device 300 can include additional elements, components, and features that may provide conventional functionality that need not be described herein. Moreover, an embodiment of the sensor device 300 can include alternative elements, components, and features if so desired, as long as the intended and described functionality remains in place.

The embodiment of the sensor device 300 shown in FIG. 5 generally includes, without limitation: the housing 304; an electronics assembly 306 located within the housing 304; a sensor interface 314; an analyte sensor 318 coupled to or included with the sensor interface 314; and an adhesive patch 322 that secures the housing 304 to the body part 302 of the user. The housing 304 is suitably shaped, sized, and configured to house or support the electronics assembly 306, the sensor interface 314, the analyte sensor 318, and other components (not shown). FIG. 5 depicts the sensor device 300 after it has been deployed and affixed to the body part 302 of the user. Accordingly, the distal end 326 of the analyte sensor 318 is depicted in its inserted position in the body part 302.

The electronics assembly 306 can be implemented as a printed circuit board to carry electronic components and elements that support operation of the sensor device 300, e.g., any number of discrete or integrated devices, electrical conductors or connectors, and the like. For example, the electronics assembly 306 may include the following items, without limitation: at least one processor 330; memory 334 to store data, processor-readable program instructions, and the like; a wireless communication interface 338 to wirelessly receive and/or transmit signals, data, instructions, and the like; a battery 342 or other power source; and an accelerometer 346 to measure acceleration, motion, and/or orientation of the sensor device 300. While FIG. 5 is described with accelerometer 346, it should be noted that the sensor device 300 can include any motion sensor (e.g., one or more of a gyroscope, a magnetometer, a gravimeter, a global positioning system unit, IMU). The electronics assembly 306 (or the components of the electronics assembly 306) can be electrically coupled to other elements of the sensor device 300 as needed to support the operation of the sensor device 300. For example, the electronics assembly 306 can be electrically coupled to at least the sensor interface 314. It should be appreciated that electrical connections to the electronics assembly 306 can be direct or indirect if so desired. Moreover, one or more components of the electronics assembly 306 may support wireless data communication internal to the sensor device 300.

The proximal end 350 of the analyte sensor 318 is electrically couplable to the sensor interface 314 to establish electrical connectivity between conductors of the analyte sensor 318 and the electronics assembly 306. Although depicted as a separate block in FIG. 5, the sensor interface 314 may be implemented on the electronics assembly 306 such that the analyte sensor 318 is electrically couplable to the electronics assembly 306 in a direct manner.

Referring again to the electronics assembly 306, each processor 330 may be implemented or performed with a general purpose processor, a content addressable memory, a microcontroller unit, a digital signal processor, an application specific integrated circuit, a field programmable gate array, any suitable programmable logic device, discrete gate or transistor logic, discrete hardware components, or any combination designed to perform the functions described here. Moreover, a processor 330 or a plurality of processors 330 may be implemented as a combination of computing devices, e.g., a combination of a digital signal processor and a microprocessor, a plurality of microprocessors, one or more microprocessors in conjunction with a digital signal processor core, or any other such configuration. In certain embodiments, one or more processors 330 of the sensor device 300 can be used to implement the at least one data processing system and/or processor 118 (depicted in FIG. 1).

The memory 334 may be realized as at least one memory element, device, or unit, such as: RAM memory, flash memory, EPROM memory, EEPROM memory, registers, a hard disk, a removable disk, optically readable media, or any other form of storage medium known in the art. In this regard, the memory 334 can be coupled to the at least one processor 330 such that the at least one processor 330 can read information from, and write information to, the memory 334. In the alternative, the memory 334 may be integral to the at least one processor 330. As an example, the at least one processor 330 and the memory 334 may reside in an ASIC. At least a portion of the memory 334 can be realized as a computer storage medium that is operatively associated with the at least one processor 330, e.g., a tangible, non-transitory computer-readable medium having computer-executable instructions stored thereon. The computer-executable instructions are configurable to be executed by the at least one processor 330 to cause the at least one processor 330 to perform certain tasks, operations, functions, and processes that are specific to the particular embodiment. In this regard, the memory 334 may represent one suitable implementation of such computer-readable media.

The wireless communication interface 338 facilitates data communication between the sensor device 300 and other components as needed during the operation of the sensor device 300. In the context of this description, the wireless communication interface 338 can be employed to transmit or receive device-related control data, sensor data obtained in connection with operation of the analyte sensor 318, data or output provided by the accelerometer 346, device-related status or operational data, calibration data, and the like. It should be appreciated that the particular configuration and functionality of the wireless communication interface 338 can vary depending on the hardware platform and specific implementation of the sensor device 300. An embodiment of the sensor device 300 may support wireless data communication using various data communication protocols. For example, the wireless communication interface 338 could support one or more wireless data communication protocols, techniques, or methodologies, including, without limitation: RF; IrDA (infrared); Bluetooth; BLE; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Although not separately shown, embodiments of the sensor device 300 may also support one or more wired/cabled data communication protocols, including, without limitation: Ethernet; powerline; home network communication protocols; USB; IEEE 1394 (Firewire); hospital network communication protocols; and proprietary data communication protocols.

In accordance with certain embodiments, the sensor device 300 communicates sensor glucose values to a destination device, such as an insulin infusion device, based on a designated schedule (for example, every five minutes, every minute, etc.). As mentioned above, communication of sensor glucose data is typically carried out wirelessly, e.g., via a BLUETOOTH wireless communication link using the wireless communication interface 338. For each sampling time, the sensor device 300 can send the most recent sensor glucose value with or without one or more historical sensor glucose values. For example, every five minutes, the sensor device 300 may send any number of sensor glucose values that have been obtained and collected during the last five-minute interval. As another example, every five minutes, the sensor device 300 may send sensor glucose values that have been obtained and collected during the last 15 minutes, for purposes of redundancy or error checking.

The battery 342 may be a single-use (disposable) battery or a rechargeable battery. The battery 342 represents the main power source of the sensor device 300, and is suitably configured to provide operating power to the at least one processor 330, the memory 334, the wireless communication interface 338, the accelerometer 346, and other components (not shown) as needed. Accordingly, the battery 342 may provide a relatively low amount of operating power during a standby or dormant state, and an increased amount of operating power during an active or deployed state (e.g., in response to processor(s) detecting deployment based on output from accelerometer 346). For example, the battery 342 may provide sufficient power to the accelerometer 346 to support its operation during a standby or dormant state, such that the accelerometer 346 can respond to acceleration or movement of the sensor device 300, e.g., when the user is ready to deploy the sensor device 300 onto the body. Moreover, the wireless communication interface 338 can be held in a low power or “off” mode during the standby or dormant state, and switched to a high power or “on” mode for operation during the active state (e.g., after deployment).

The accelerometer 346 is configured, arranged, and operated to measure acceleration, motion, orientation, or handling of the host device, e.g., the sensor device 300. In certain embodiments, the accelerometer 346 is configured to generate a suitably formatted output (e.g., a signal, a voltage level, an acceleration measurement or reading, a wakeup signal, a flag, a logical state, or the like) in response to detecting acceleration of the sensor device 300. Alternatively or additionally, the accelerometer 346 can be polled by the at least one processor 330 to obtain acceleration readings on demand, as needed, according to a designated schedule, periodically, etc. Other methodologies for detecting or measuring acceleration may be employed by embodiments of the host device. The accelerometer 346 can be implemented using any type of commercially available or custom accelerometer device, circuit, or package.

When the sensor device 300 or the accelerometer 346 is enabled or in the active state, the sensor device 300 may communicate the accelerometer output to a destination device (such as a remote processing device or system or a user device) more often than the sensor glucose values. Indeed, the accelerometer output should be communicated and handled at a high sampling rate to enable the system to quickly detect and respond to motion, movement, and acceleration of the sensor device 300 in real time or substantially real time. To this end, accelerometer output may be communicated every second, every 500 milliseconds, or the like. In accordance with certain embodiments, the accelerometer data (3-axes) is collected at a frequency of 200 Hz, and at a resolution of 12 bits. When streaming accelerometer data to a mobile device, a BLUETOOTH wireless link changes to a high throughput configuration to accommodate 1,400 byes per second. For this implementation, the latency from collection to transmission is less than 500 milliseconds. This accelerometer data stream can be compressed to 900 bytes per second (or less), depending on the particular embodiment.

The at least one processor 330 controls certain operations of the sensor device 300. In particular, the at least one processor 330 is configured and arranged to receive the output generated by the accelerometer 346, process the output, and initiate communication of the output (as needed) to one or more other components of the system 100. For example, the output of the accelerometer 346 can be wirelessly transmitted to the user device 106 for processing and handling as described in more detail below.

In certain embodiments, the accelerometer 346 can be operated in a low power mode (e.g., a wake-on-motion mode) and a normal power mode. For example, the accelerometer 346 may be held in the low power mode (to conserve battery power) until the user activates the sensor device 300 or until a threshold amount of motion or acceleration is detected. The processor(s) 330 may also be placed into a low-power mode to conserve power. When motion or a certain motion pattern is detected, the accelerometer 346 and/or the processor 330 may switch into a higher power mode. Conversely, the accelerometer 346 can be disabled or returned to its low power mode after deployment of the sensor device 300.

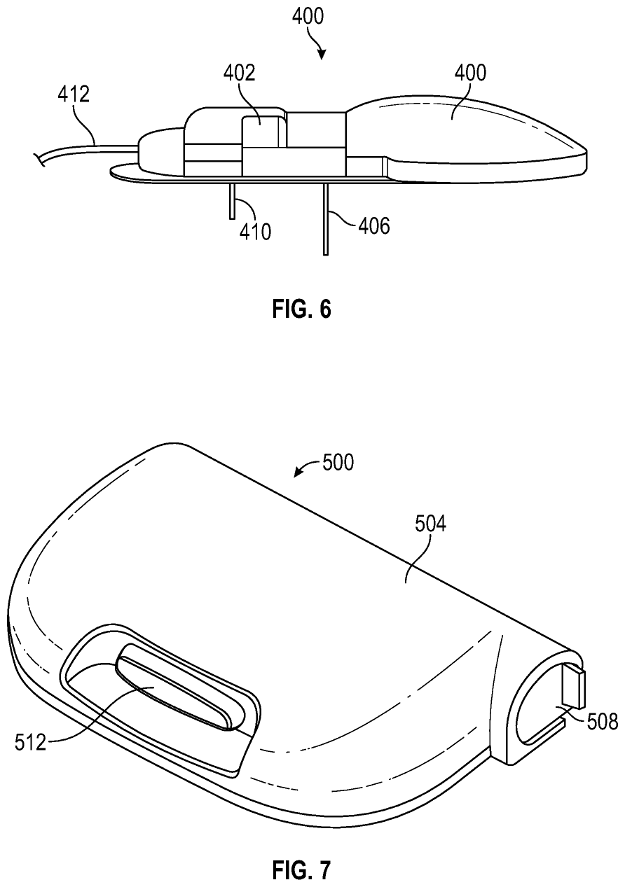

FIG. 6 is a side perspective view of an embodiment of a user-mountable device 400 implemented as an analyte sensor unit 402 combined with a fluid infusion set 404. The device 400 is one suitable implementation of the user-mountable electronic device or assembly 102 shown in FIG. 1. As described above with reference to the sensor device 300, the analyte sensor unit 402 has an analyte sensor 406 that is insertable into the body of the user. The fluid infusion set 404 includes a fluid conduit 410 that is insertable into the body of the user. The fluid conduit 410 is fluidly connected to a fluid delivery tube 412, such that medication fluid carried by the fluid delivery tube 412 flows into the body via the fluid conduit 410. The device 400 can be deployed onto the body of the user in the manner described above. Moreover, the device 400 may include the components shown in FIG. 5 to support the accelerometer-based features and functions described below.

FIG. 7 is a top perspective view of an embodiment of a user-mountable device 500 implemented as a patch pump device that is suitable for use as an insulin delivery system. The device 500 is one suitable implementation of the user-mountable electronic device or assembly 102 shown in FIG. 1. The device 500 can be implemented as a combination unit that includes an insertable insulin delivery cannula and an insertable glucose sensor (both of which are hidden from view in FIG. 7). The device 500 includes a housing 504 that serves as a shell for a variety of internal components. FIG. 7 shows the device 500 with a removable fluid cartridge 508 installed and secured therein. The housing 504 is suitably configured to receive, secure, and release the removable fluid cartridge 508. The device 500 includes at least one user interface feature, which can be actuated by the patient as needed. The illustrated embodiment of the device 500 includes a button 512 that is physically actuated. The button 512 can be a multipurpose user interface if so desired to make it easier for the user to operate the device 500. In this regard, the button 512 can be used in connection with one or more of the following functions, without limitation: waking up the processor and/or electronics of the device 500; triggering an insertion mechanism to insert a fluid delivery cannula and/or an analyte sensor into the subcutaneous space or similar region of the user; configuring one or more settings of the device 500; initiating delivery of medication fluid from the fluid cartridge 508; initiating a fluid priming operation; disabling alerts or alarms generated by the device 500; and the like. In lieu of the button 512, the device 500 can employ a slider mechanism, a pin, a lever, a switch, a touch-sensitive element, or the like. In certain implementations, the device 500 can be deployed onto the body of the user in the manner described above. Moreover, the device 500 may include the components shown in FIG. 5 to support the accelerometer-based features and functions described below.

The user-mountable electronic devices shown in FIGS. 1-7 and described above are non-limiting examples. The device configurations and device deployment guidance methodologies described herein can be utilized with other user-mountable devices if so desired. Moreover, the device configurations and device deployment guidance methodologies described herein can be utilized in the context of non-medical systems and applications, or in the context of medical systems and applications that are unrelated to the treatment of diabetes.

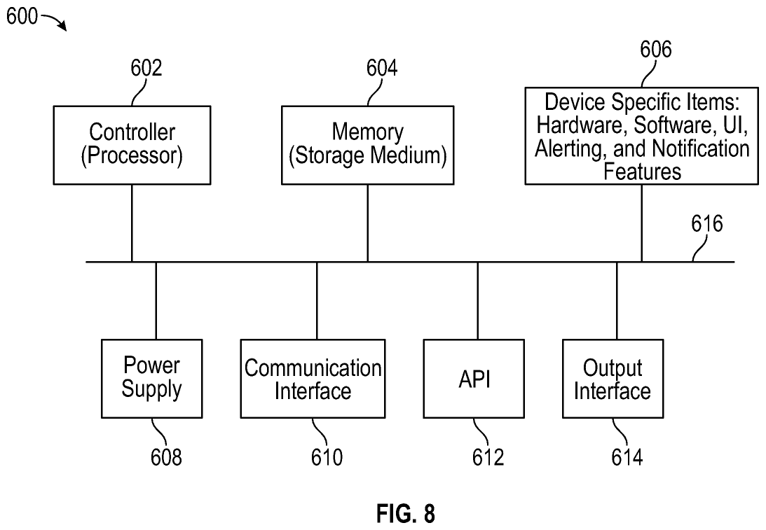

In accordance with certain embodiments, any or all of the components shown in FIGS. 1-7 can be implemented as a computer-based or a processor-based device, system, or component having suitably configured hardware and software written to perform the functions and methods needed to support the features described herein. In this regard, FIG. 8 is a simplified block diagram representation of an exemplary embodiment of a computer-based or processor-based device 600 that is suitable for deployment in the system 100 shown in FIG. 1.

The illustrated embodiment of the device 600 depicted in FIG. 8 is intended to be a high-level and generic representation of one suitable platform. In this regard, any computer-based or processor-based component of the system 100 can utilize the architecture of the device 600. The illustrated embodiment of the device 600 generally includes, without limitation: at least one controller (or processor) 602; a suitable amount of memory 604 that is associated with the at least one controller 602; device-specific items 606 (which may include, without limitation: hardware, software, firmware, user interface (UI), alerting, and notification features); a power supply 608 such as a disposable or rechargeable battery; a communication interface 610; at least one application programming interface (API) 612; and an output interface 614, such as a display element. Of course, an implementation of the device 600 may include additional elements, components, and functionality configured to support various features that are unrelated to the primary subject matter described here. For example, the device 600 may include certain features and elements to support conventional functions that might be related to the particular implementation and deployment of the device 600. In practice, the elements of the device 600 may be coupled together via at least one bus or any suitable interconnection architecture 616.

The at least one controller 602 may be implemented or performed with a general purpose processor, a content addressable memory, a microcontroller unit, a digital signal processor, an application specific integrated circuit, a field programmable gate array, any suitable programmable logic device, discrete gate or transistor logic, discrete hardware components, or any combination designed to perform the functions described here. Moreover, the at least one controller 602 may be implemented as a combination of computing devices, e.g., a combination of a digital signal processor and a microprocessor, a plurality of microprocessors, one or more microprocessors in conjunction with a digital signal processor core, or any other such configuration.

The memory 604 may be realized as at least one memory element, device, or unit, such as: RAM memory, flash memory, EPROM memory, EEPROM memory, registers, a hard disk, a removable disk, a CD-ROM, or any other form of storage medium known in the art. In this regard, the memory 604 can be coupled to the at least one controller 602 such that the at least one controller 602 can read information from, and write information to, the memory 604. In the alternative, the memory 604 may be integral to the at least one controller 602. As an example, the at least one controller 602 and the memory 604 may reside in an ASIC. At least a portion of the memory 604 can be realized as a computer storage medium that is operatively associated with the at least one controller 602, e.g., a tangible, non-transitory computer-readable medium having computer-executable instructions stored thereon. The computer-executable instructions are configurable to be executed by the at least one controller 602 to cause the at least one controller 602 to perform certain tasks, operations, functions, and processes that are specific to the particular embodiment. In this regard, the memory 604 may represent one suitable implementation of such computer-readable media. Alternatively or additionally, the device 600 could receive and cooperate with computer-readable media (not separately shown) that is realized as a portable or mobile component or platform, e.g., a portable hard drive, a USB flash drive, an optical disc, or the like.

The device-specific items 606 may vary from one embodiment of the device 600 to another. For example, the device-specific items 606 will support: sensor device operations when the device 600 is realized as a sensor device; smartphone features and functionality when the device 600 is realized as a smartphone; activity tracker features and functionality when the device 600 is realized as an activity tracker; smart watch features and functionality when the device 600 is realized as a smart watch; medical device features and functionality when the device is realized as a medical device; etc. In certain cases, the device-specific items 606 includes the at least one sensor device 104 (see FIG. 1). In practice, certain portions or aspects of the device-specific items 606 may be implemented in one or more of the other blocks depicted in FIG. 8.

If present, the UI of the device 600 may include or cooperate with various features to allow a user to interact with the device 600. Accordingly, the UI may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the device 600. The UI may include one or more graphical user interface (GUI) control elements that enable a user to manipulate or otherwise interact with an application via the output interface 614. The output interface 614 and/or the device-specific items 606 may be utilized to generate, present, render, output, and/or annunciate alerts, alarms, messages, or notifications that are associated with operation of the system 100, associated with a status or condition of the user, associated with operation, status, or condition of the system 100, etc.

The communication interface 610 facilitates data communication between the device 600 and other components as needed during the operation of the device 600. In the context of this description, the communication interface 610 can be employed to transmit or stream device-related control data, patient-related user status, device-related status or operational data, sensor data, accelerometer data, calibration data, and the like. It should be appreciated that the particular configuration and functionality of the communication interface 610 can vary depending on the hardware platform and specific implementation of the device 600. In practice, an embodiment of the device 600 may support wireless data communication and/or wired data communication, using various data communication protocols. For example, the communication interface 610 could support one or more wireless data communication protocols, techniques, or methodologies, including, without limitation: RF; IrDA (infrared); Bluetooth; BLE; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Moreover, the communication interface 610 could support one or more wired/cabled data communication protocols, including, without limitation: Ethernet; powerline; home network communication protocols; USB; IEEE 1394 (Firewire); hospital network communication protocols; and proprietary data communication protocols.

The at least one API 612 supports communication and interactions between software applications and logical components that are associated with operation of the device 600. For example, one or more APIs 612 may be configured to facilitate compatible communication and cooperation with the patient care application 108, and to facilitate receipt and processing of data from sources external to the device 600 (e.g., databases or remote devices and systems).

The output interface 614 is suitably configured to enable the device 600 to render and display various screens, recommendation messages, alerts, alarms, notifications, GUIs, GUI control elements, drop down menus, auto-fill fields, text entry fields, message fields, or the like. Of course, the output interface 614 may also be utilized for the display of other information during the operation of the device 600, as is well understood. Notably, the specific configuration, operating characteristics, size, resolution, and functionality of a display element can vary depending upon the implementation of the device 600.

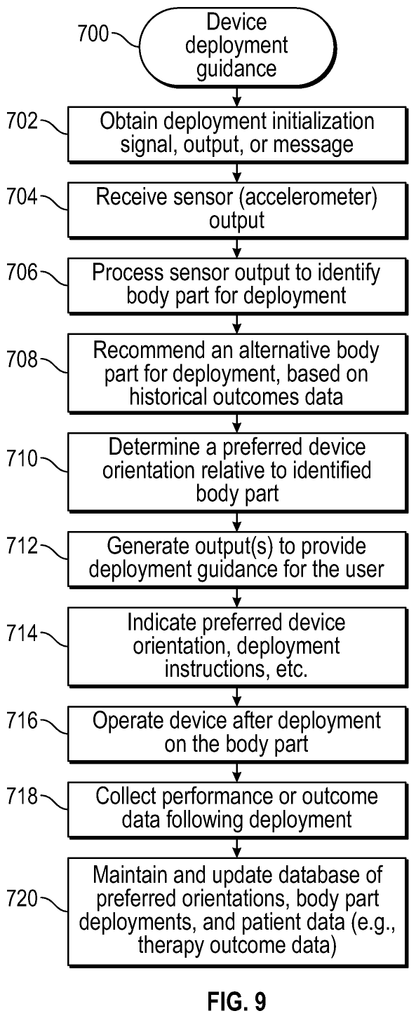

FIG. 9 is a flow chart that illustrates an embodiment of a device deployment guidance process 700. It should be appreciated that an implementation of the process 700 may include any number of additional or alternative tasks, the tasks shown in FIG. 9 need not be performed in the illustrated order, and the process 700 may be incorporated into a more comprehensive procedure or process having additional functionality not described in detail herein. Moreover, one or more of the tasks shown in FIG. 9 could be omitted from an embodiment of the process 700 as long as the intended intermediate or overall functionality remains intact.

The process 700 may begin in response to an initialization, startup, or triggering event. In this regard, the process 700 may obtain an initialization signal, output, or message indicating that the user intends to deploy a user-mountable electronic device (task 702). The initialization signal, output, or message may be user-initiated at a device within the system environment. Alternatively or additionally, the initialization signal, output, or message may be automatically generated and provided in response to detection of a particular event, condition, or status of the user-mountable electronic device. For example, the accelerometer or another sensor device onboard the user-mountable electronic device may generate output that indicates the user is handling, moving, or otherwise preparing the user-mountable electronic device for deployment onto the body.

This description assumes that an obtained deployment initialization signal, output, or message triggers the remainder of the process 700. Accordingly, the process 700 may proceed by receiving sensor output generated by at least one sensor device onboard the user-mountable electronic device (task 704). For this example, the at least one sensor device is an accelerometer device, and the received sensor output is the data or output provided by the accelerometer device, but the at least one sensor device may be any motion sensor as described above. The received sensor output is processed to identify a body part on which the user intends to deploy the user-mountable electronic device (task 706). In certain embodiments, received accelerometer data can be analyzed and compared (in real-time or substantially real-time) to historical device deployment data to determine whether the user-mountable electronic device is being held, positioned, moved, adjusted, and/or located in a manner that corresponds to a known body part. Alternatively or additionally, the process 700 may consider additional information or data from other sources when identifying the body part for deployment. For example, the user may identify the body part by interacting with the user device, the user-mountable electronic device, a web page, or the like. As another example, audio, image, and/or video data (captured while the user is handling the user-mountable electronic device) may be considered to determine the body part.

In certain embodiments, the received accelerometer data need not be analyzed or processed in real-time or substantially real-time. Instead, the received accelerometer data can be collected for post-processing or post-analysis, for purposes of informing subsequent device deployment operations. For example, received accelerometer data can be collected as a way to automatically log or record different wear locations for a user, such that historical wear locations can be identified for future reference or deployment guidance. In such an example, process 700 may begin at task 704 or 706.

In accordance with some examples, the process 700 can determine one or more alternative body parts (other than the body part identified at task 706), recommend the one or more alternative body parts for deployment, and cause an output interface to provide guidance to deploy the user-mountable electronic device on the one or more alternative body parts (task 708). An alternative body part can be recommended based on historical outcomes data for medical devices that have been deployed on particular body parts. The historical outcomes data may include outcomes data for the user and/or outcomes data associated with a population of users (i.e., data for at least one person other than the user). For example, the process 700 may suggest deployment of the device on the right thigh instead of the left upper arm, based on collected patient data that indicates better therapy outcomes when the device is located on the right thigh.

The following description assumes that the user will deploy the device on the body part identified at task 706. The process 700 continues by determining at least one preferred orientation of the user-mountable electronic device relative to the identified body part (task 710). Device orientation can be detected and recorded based on real-time (or substantially real-time) accelerometer data. In this regard, device orientation may be determined relative to a gravity vector as measured or observed based on the accelerometer data. Preferred device orientations for different body parts can be maintained in an appropriate database, e.g., the database(s) 114 (see FIG. 1). In this regard, the database may include a number of database objects, including preferred orientations for user-mountable electronic devices and for different body part deployments for the user-mountable electronic devices. Accordingly, task 710 may be carried out by accessing such a database to find preferred device orientations for the identified body part and/or one or more alternative body parts. For example, if the identified body part is the user's forearm, a preferred device orientation may call for deployment of the device such that its major longitudinal axis corresponds to the length of the forearm. Conversely, the preferred device orientation may call for deployment of the device such that its major longitudinal axis is generally perpendicular to the length of the forearm. As another example, if the identified body part is the user's abdomen, a preferred device orientation may call for deployment of the device such that it's major longitudinal axis is substantially parallel to the waistline, or is rotated by a specified amount (in degrees) relative to the waistline.

The process 700 may continue by causing an output interface to generate and provide deployment guidance that indicates the preferred orientation of the user-mountable electronic device (task 712). In accordance with certain implementations, the output interface includes a display element of a user device, and the process 700 causes the display device to display instructions regarding how to position the user-mountable electronic device on the identified body part prior to deployment (task 714). As another example, the process 700 causes the display device to display at least one image, at least one video clip, or animated content that shows how to position the user-mountable electronic device on the identified body part prior to deployment (task 714). In accordance with certain embodiments, post-deployment data is collected over a period of time to record historical deployment locations (body parts) and/or historical device orientations, and the process 700 causes the display device to display a picture or a diagram of a body or a body part with markers or indicators corresponding to previous device deployment locations and/or orientations. Video or image content may be in the form of saved tutorials, or it can include augmented reality features that leverage the camera functions of a user device and/or the accelerometer output as generated during the device deployment routine.

In certain scenarios, the process 700 need not identify a preferred device deployment orientation. For example, if the system does not have sufficient historical outcomes data to recommend a specific device orientation, then the process 700 may simply provide general guidance or instructions for deploying the user-mountable device onto an identified body part. As another example, the system may have enough historical outcomes data to determine that device orientation has little to no impact on the patient outcome. Accordingly, the process 700 may provide general guidance or deployment instructions and/or indicate that the user is free to choose the device orientation.

In some examples, the process 700 assumes that the user-mountable device is successfully deployed onto the identified body part, and in a known orientation that can be recorded or otherwise documented. The user-mountable device is operated after deployment on the particular body part, and in the specific orientation relative to the particular body part (task 716). In other examples, the process 700 continues by collecting performance or outcome data for the user-mountable device following deployment on the particular body part (task 718). The performance or outcome data can originate from the user-mountable device itself, from a sensor or other component of the user-mountable device, from a related monitoring device or system, from a remote server device or system, from a user device, or the like. The collected performance or outcome data can be correlated with the particular body part and the specific device orientation, for archiving or for use during subsequent device deployments. In this regard, a suitably configured and formatted database, such as the database(s) 114 described above with reference to FIG. 1, can be maintained in connection with user-mountable device deployments (task 720). The maintained database(s) can include preferred orientations for different user-mountable electronic devices, and for different body part deployments. As mentioned above, the maintained database(s) can identify preferred body parts for deployment and preferred device orientations for the identified body parts. Moreover, the information maintained in the database(s) may be user-specific (i.e., based on historical outcome data from the user) or user-agnostic (i.e., based on historical outcome data from a plurality of different users, which may or may not include the user of interest).

In most situations, the at least one sensor device (e.g., an accelerometer device) is located within the housing of the user-mountable electronic device. Alternatively, the at least one sensor device can be located in the deployment mechanism (which is usually removed/discarded after it has been actuated to deploy the user-mountable device). The following description assumes that the at least one sensor device is located at the user-mountable electronic device, and that the at least one sensor device remains active and operational after deployment of the user-mountable electronic device. Consequently, the user-mountable electronic device can leverage the output of the at least one sensor device for other purposes and applications if so desired.

In accordance with one non-limiting example, after deployment of the user-mountable electronic device on the body of the user, the at least one sensor device generates sensor output in response to physical tapping, bumping, shock, or concussion on the housing of the device. The user-mountable electronic device processes this sensor output to detect at least one distinguishable tapping gesture, input, or pattern resulting from the user tapping on or otherwise interacting with the housing. If a known tapping action is detected, then the user-mountable electronic device can perform one or more operations or functions (as appropriately linked to the detected tapping action). If not, then the user-mountable electronic device can disregard or ignore the tapping action, and need not respond to the tapping action. In certain implementations, the user-mountable electronic device is configured to detect a plurality of different tapping patterns corresponding to a plurality of different operations, functions, or processes performed by the user-mountable electronic device. Moreover, at least some of the plurality of different tapping patterns can be user-configurable, such that the user can program or train the user-mountable electronic device to react to customized user-designated tapping inputs (e.g., tap patterns or combinations).

Any number of device features, functions, or operations can be controlled by tapping actions detected by processing the sensor output. In accordance with some exemplary embodiments where the user-mountable device is a continuous glucose monitor device, some or all of the following operations can be linked to corresponding tapping actions (e.g., tap patterns or combinations): activate device; deactivate device; wireless pairing with another device; re-initiate wireless connection with another device; enable communication from the user-mountable device to a third party device or system (e.g., a caregiver system); control setup or initialization of a newly deployed user-mountable device (e.g., a replacement continuous glucose monitor device); meal announcements; silence, mute, or snooze an alert or alarm or notification; power management; detect removal or loss of the user-mountable electronic device; detect injury to the user (e.g., falling down, accidents, physical shock or impact); detect impact or damage to the user-mountable electronic device; user activity/sleep detection and characterization; detect user body or limb movements and gestures; detect meal consumption; detect or measure respiratory rate.

The specific types and number of operations and functions linked to tapping actions may vary from one embodiment to another, and will be appropriate to the particular type of user-mounted electronic device. The examples provided above are contextually relevant where the device is a continuous glucose monitor. Additional, alternative, and different operations, features, and functions can be supported where the device something other than a continuous glucose monitor, e.g., a fluid infusion device, an activity monitor, or the like.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A system comprising:
a user-mountable electronic device comprising:
a housing; and
at least one sensor device located within the housing and configured to generate sensor output that indicates orientation or motion of the user-mountable electronic device;
an output interface; and
at least one processor to:
receive the sensor output generated by the at least one sensor device;
identify, based on the received sensor output, a body part on which the user intends to deploy the user-mountable electronic device;
determine a preferred orientation of the user-mountable electronic device relative to the identified body part; and
cause the output interface to provide deployment guidance that indicates the preferred orientation of the user-mountable electronic device.

2. The system of claim 1, wherein the output interface comprises a display element or an audio transducer of a user device.

3. The system of claim 1, wherein the output interface comprises a display element or an audio transducer of the user-mountable electronic device.

4. The system of claim 1, wherein the at least one sensor device comprises an accelerometer.

5. The system of claim 1, wherein the at least one sensor device comprises an inertial measurement unit.

6. The system of claim 1, wherein the at least one sensor device comprises a gyroscope.

7. The system of claim 1, wherein the at least one sensor device comprises a magnetometer.

8. The system of claim 1, wherein the at least one processor is located at a computer-based device other than the user-mountable electronic device.

9. The system of claim 1, wherein the user-mountable electronic device comprises at least one body-insertable element.

10. The system of claim 1, wherein the user-mountable electronic device comprises a continuous glucose monitor having a glucose sensor.

11. The system of claim 10, wherein:
after deployment of the user-mountable electronic device on the body of the user, the at least one sensor device generates sensor output in response to physical tapping on the housing;
the user-mountable electronic device processes the sensor output to detect at least one distinguishable tapping gesture, input, or pattern resulting from the user tapping on the housing; and
the user-mountable electronic device performs at least one operation that is linked to the detected at least one distinguishable tapping gesture, input, or pattern.

12. The system of claim 11, wherein the user-mountable electronic device is configured to detect a plurality of different tapping patterns corresponding to a plurality of different operations, functions, or processes performed by the user-mountable electronic device.

13. The system of claim 12, wherein at least some of the plurality of different tapping patterns are user-configurable.

14. The system of claim 1, wherein the user-mountable electronic device comprises a fluid infusion device.

15. The system of claim 1, wherein the user-mountable electronic device comprises a medication fluid infusion set having a body-insertable fluid conduit.

16. The system of claim 1, further comprising a database that includes preferred orientations for user-mountable electronic devices and for different body part deployments.

17. The system of claim 1, wherein the at least one processor determines an alternative body part, other than the identified body part, and causes the output interface to provide guidance to deploy the user-mountable electronic device on the alternative body part.

18. A method comprising:
receiving, from at least one sensor device of a user-mountable electronic device, sensor output that indicates orientation or motion of the user-mountable electronic device;
identifying, based on the received sensor output, a body part on which the user intends to deploy the user-mountable electronic device;
determining a preferred orientation of the user-mountable electronic device relative to the identified body part; and
causing an output interface to provide deployment guidance that indicates the preferred orientation of the user-mountable electronic device.

19. The method of claim 18, wherein:
the output interface comprises a display element of a user device; and
the method causes the display device to display instructions regarding how to position the user-mountable electronic device on the identified body part prior to deployment.

20. The method of claim 18, wherein:
the output interface comprises a display element of a user device; and
the method causes the display device to display at least one image, at least one video clip, or animated content that shows how to position the user-mountable electronic device on the identified body part prior to deployment.

21. The method of claim 18, further comprising:
obtaining an initialization signal, output, or message indicating that the user intends to deploy the user-mountable electronic device, wherein the receiving step, the identifying step, the determining step, and the causing step are triggered by the obtained initialization signal, output, or message.

22. The method of claim 18, further comprising:
maintaining a database that includes preferred orientations for user-mountable electronic devices and for different body part deployments.

23. The method of claim 18, wherein:
the user-mountable electronic device comprises a medical device for the user;
the medical device is operated after deployment on a particular body part and in a specific orientation relative to the particular body part; and
the method further comprises the steps of:
collecting performance or outcome data for the medical device following deployment on the particular body part; and
correlating the performance or outcome data with the particular body part and the specific orientation.

24. The method of claim 18, further comprising the step of:
causing the output interface to identify a recommended body part for deployment of the user-mountable electronic device, based on historical outcomes data.

25. The method of claim 24, wherein the historical outcomes data includes outcomes data for the user.

26. The method of claim 24, wherein the historical outcomes data includes outcomes data for at least one person other than the user.

27. The method of claim 18, wherein the output interface provides the deployment guidance in real-time or substantially real-time in response to sensor output received from the at least one sensor device during deployment of the user-mountable electronic device.

28. The method of claim 18, wherein:
the deployment guidance is generated in response to historical sensor output collected from one or more previously deployed user-mountable electronic devices or assemblies; and
the output interface provides the deployment guidance during deployment of the user-mountable electronic device.

29. A method comprising:
receiving, from at least one sensor device of a first user-mountable electronic device, sensor output that indicates orientation or motion of the first user-mountable electronic device, and corresponding performance data for the first user-mountable device;
identifying, based on the received sensor output and corresponding performance data, a preferred body part or orientation for deployment of a second user-mountable electronic device; and
causing an output interface to provide deployment guidance that indicates the preferred body part or orientation for deployment of the second user-mountable electronic device.
